(19) United States
(12) Patent Application Publication
(10) Pub. No.: US 2018/0206972 A1
(43) Pub. Date: Jul. 26, 2018

(54) GRAFT PROSTHESIS COUPLER, MODULAR SYSTEM, AND METHODS OF USE

(71) Applicant: Bolton Medical, Inc., Sunrise, FL (US)

(72) Inventors: Samuel Arbefeuille, Sunrise, FL (US); Eduardo Alejandro Garcia, Sunrise, FL (US); Timothy Lostetter, Sunrise, FL (US)

(21) Appl. No.: 15/672,404

(22) Filed: Aug. 9, 2017

Related U.S. Application Data
(60) Provisional application No. 62/372,882, filed on Aug. 10, 2016.

(51) Int. Cl. A61F 2/06; A61F 2/07; A61F 2/848; A61F 2/852

(52) U.S. Cl. CPC A61F 2/064; A61F 2/07; A61F 2002/067; A61F 2/852; A61F 2002/8483; A61F 2/848

(57) ABSTRACT

A graft prosthesis coupler includes a plurality of luminal grafts, each of which defines a first end, and a second end and a major longitudinal axis, the major longitudinal axes being substantially parallel to each other. The graft prosthesis coupler also includes a circumferential graft that has first and second ends and extends about the plurality of luminal grafts, and a first end wall that lies in a surface defined by the first ends of the luminal grafts and the first end of the circumferential graft. In another embodiment, a graft prosthesis coupler includes a plurality of luminal grafts attached to each other. A modular graft system includes the graft prosthesis coupler and branch graft prostheses. The graft prosthesis coupler can be implanted in a patient at a site of an aneurysm to facilitate implanting the branch graft prostheses into branch vessels at the site of the aneurysm.

### RELATED APPLICATION

This application claims the benefit of U.S. Provisional Application No. 62/372,882, filed on Aug. 10, 2016. The entire teachings of the above application are incorporated herein by reference.

### BACKGROUND

Aortic pathologies, including aortic aneurysms, can be treated by open surgical reconstruction, or alternatively, endovascular repair, which is a minimally invasive alternative to open surgical repair. Optimizing a successful outcome of endovascular repair, however, requires assessment of the patient's anatomy and, in the case of an aortic aneurysm, an appropriate stent graft that spans the proximal and distal ends of the aneurysm to insure complete exclusion of the aneurysm sac, anchoring of the stent graft in the aorta, and minimal endoleaks. Endoleaks and post-surgical enlargement of the aneurysm site can require additional repair to seal any expansion of the aneurysm sac, and, generally, must be done without significantly compromising blood flow through the surgical site to surrounding viscera and associated structures.

Therefore, a need exists for new and improved endovascular repair devices and methods to treat aortic pathologies, in particular aortic aneurysms.

### SUMMARY OF THE INVENTION

The present invention relates to prostheses for use in treating and repairing aortic vascular damage, such as vascular damage associated with an abdominal aortic aneurysm (AAA), dissections and penetrating ulcers, in particular when the vascular damage is associated with aortic damage at the level of the hypogastric artery.

In an embodiment, the invention is a graft prosthesis coupler that includes a plurality of luminal grafts, each of which defines a first end, a second end, an outside surface between the first end and the second end, and a longitudinal axis extending between the first end and the second end of each of the plurality of luminal grafts, the longitudinal axes being substantially parallel to each other. A circumferential graft extends about and secures the plurality of luminal grafts relative to each other, and includes a first end and a second end. A first end wall lies in a surface defined by the first ends of the plurality of luminal grafts and the first ends of the circumferential graft.

In another embodiment, the invention is a modular stent graft prosthesis system, such as a kit, that includes a stent graft, such as a bifurcated stent graft, a graft prosthesis coupler and a plurality of branch grafts. In the case of a bifurcated stent graft, the bifurcated stent graft includes a main stent graft component, a first leg extending from the main stent graft component, and a second leg extending from the main stent graft component that is substantially parallel to the first leg. The graft prosthesis coupler includes a plurality of luminal grafts, each of which defines a first end, a second end, and a longitudinal axis extending between the first end and the second end. The longitudinal axes of the luminal grafts are substantially parallel to each other. A circumferential graft extends about and, in one embodiment, secures the plurality of luminal grafts relative to each other. The circumferential graft has a first end and a second end. A first end wall lies in a surface defined by first ends of the plurality of luminal grafts and the first end of the circumferential graft. Each of the plurality of branch grafts is conformable to one of the plurality of luminal grafts of the graft prosthesis coupler.

In a further embodiment, the invention is a modular stent graft prosthesis system, such as a kit, that includes a graft prosthesis coupler and a plurality of branch grafts. The graft prosthesis coupler includes a plurality of luminal grafts, each of which defines a first end and a second end. Each of the luminal grafts defines a longitudinal axis between each of the first end and the second end of the plurality of luminal grafts, the longitudinal axes being substantially parallel to each other. A circumferential graft extends about the plurality of luminal grafts, and has a first end and a second end. A first end wall lies in a surface defined by the first ends of the plurality of luminal grafts and the first end of the circumferential graft. Each of the plurality of branch stent grafts is conformable to at least one of the plurality of luminal grafts.

In yet another embodiment, the invention is a graft prosthesis coupler that includes a plurality of luminal grafts, each of which defines a first end and a second end and a longitudinal axis, the longitudinal axes being substantially parallel to each other, and wherein the plurality of luminal grafts are attached to each other, the luminal grafts together defining a first end and a second end of the graft prosthesis coupler and defining a central longitudinal axis. The first end and the second end can each, independently, define a plane, a concave surface, or a convex surface.

In still another embodiment, the invention is a method for implanting a modular stent graft prosthesis at an aneurysm site of a patient. In one embodiment, the method includes delivering a stent graft to an aneurysm site in a patient, the stent graft defining an open proximal end, an open distal end, and a main lumen, the main lumen extending between the open proximal end and the open distal end. A graft prosthesis coupler is inserted into the main lumen of the stent graft. The graft coupler includes a plurality of luminal grafts, each of which defines a first end, a second end, and a longitudinal axis, wherein the longitudinal axes are substantially parallel to each other. A circumferential graft of the graft prosthesis extends about the plurality of luminal grafts and, in one embodiment, secures the luminal grafts. The circumferential graft defines a first end and a second end. In this embodiment, a first end wall of the graft prosthesis coupler lies in a surface defined by the first ends of the luminal grafts and the first end of the circumferential graft. The surface can be, for example, in the shape of a concavity. Each of a plurality of branch graft prostheses is inserted into a corresponding one of the plurality of the luminal grafts of the graft prosthesis, each of the plurality of branch graft prostheses having a branch graft prosthesis open proximal end and a branch graft prosthesis open distal end, wherein the stent graft, the graft prosthesis coupler and the plurality of branch graft prostheses together make up the modular stent graft prosthesis as, for example, a kit. The distal end of each of the plurality of branch graft prostheses is inserted into a corresponding aortic branch of the patient at the aneurysm site, thereby implanting the modular stent graft prosthesis at the aneurysm site of the patient.

In another embodiment, the invention is a method of extending a stent graft prosthesis at an aneurysm site of a patient. In this embodiment, the method includes delivering a graft prosthesis coupler into a main lumen of an pre-existing luminal prosthesis in a blood vessel at an aneurysm site of a patient. The graft prosthesis coupler includes a plurality of luminal grafts, each of which defines a first end, a second end, and a longitudinal axis. The longitudinal axes of the plurality of luminal grafts are substantially parallel to each other. A circumferential graft of the graft prosthesis coupler extends about the plurality of luminal grafts and the circumferential graft has a first end and a second end. In one embodiment, the circumferential graft secures the luminal grafts. A first end wall of the graft prosthesis coupler lies in a surface, such as a concavity, defined by the first ends of the luminal grafts and the first end of the circumferential graft. Optionally, a second end wall of the graft prosthesis coupler lies in a surface, such as a plane, defined by the second ends of the luminal grafts and the second end of the circumferential graft. Each of a plurality of branch graft prostheses is inserted into a corresponding one of the plurality of luminal grafts of the graft prosthesis coupler. Each of the plurality of branch graft prostheses has a branch graft prosthesis open end and a branch graft prosthesis open distal end, wherein the graft prosthesis coupler and the plurality of branch graft prostheses collectively make up an extension, or enhancement, for a pre-existing prosthesis, such as in a kit. The distal end of each of the plurality of branch graft prostheses is inserted into a corresponding aortic branch of the patient at the aneurysm site, thereby extending, or enhancing, the stent graft prosthesis at the aneurysm site of the patient.

In still yet another embodiment of the invention, a method of implanting a modular stent graft prosthesis at an aneurysm site of a patient includes delivering a stent graft to an aneurysm site in a patient, the stent graft defining an open proximal end, an open distal end, and a main lumen, wherein the main lumen extends between the open proximal end and the open distal end. A graft prosthesis coupler is inserted into the main lumen of the stent graft, wherein the stent graft includes a plurality of luminal grafts, each of which defines a first end, a second end and a longitudinal axis. The longitudinal axes of the luminal grafts are substantially parallel to each other, and the plurality of luminal grafts are attached to each other. Each of a plurality of branch graft prostheses is inserted into a corresponding one of the plurality of luminal grafts of the graft prosthesis coupler, each of the plurality of branch graft prostheses defining a branch graft prosthesis open proximal end, a branch graft prosthesis open distal end, and lumen extending there between. The stent graft, the graft prosthesis coupler, and the plurality branch graft prostheses together make up the modular stent graft prosthesis, such as in a kit. The distal end of each of the plurality of branch graft prostheses is inserted into a corresponding aortic branch of the patient at the aneurysm site, thereby implanting the modular stent graft prosthesis at aneurysm site of the patient.

In another embodiment of the invention, a method of implanting a modular stent graft prosthesis at an aneurysm site of a patient includes delivering a stent graft through a blood vessel to an aneurysm site in a patient, the stent graft defining an open proximal end, an open distal end, and a main lumen, the main lumen extending between the open proximal end and the open distal end. A graft prosthesis coupler is inserted through the main lumen of the stent graft, the graft prosthesis coupler including a plurality of luminal grafts, each of which defining a longitudinal axis, that are substantially parallel to each other, and wherein the plurality of luminal grafts are attached to each other. Each of a plurality of branch graft prostheses are inserted into a corresponding one of the plurality of luminal grafts of the graft prosthesis coupler, each of the plurality of the branch graft prostheses having a branch graft prosthesis open proximal end and a branch graft prosthesis open distal end, the stent graft, the graft prosthesis coupler, and the plurality of branch graft prostheses together making up the modular stent graft prosthesis. The distal end of each of the plurality of branch graft prostheses is inserted into a corresponding arterial branch of the patient at the aneurysm site, thereby implanting the modular stent graft prosthesis at the aneurysm site of the patient.

The prostheses and methods of the invention have several advantages to accommodate the extent of aortic damage, and changes following implantation of a prosthesis, while preserving blood flow to associated viscera and surrounding tissues. In the case of an abdominal aortic aneurysm (AAA) repair, an understanding of the anatomy of the patient's iliac artery and its involvement in aortic damage is critical since the iliac artery is a channel for deployment of the prosthesis and the site of sealing of the distal end of the prosthesis. During AAA repair, care should be taken to avoid exclusion of both hypogastric arteries to minimize damage to surrounding tissues and viscera, such as colorectal ischemia and buttock claudication. The graft prostheses and methods of the invention can be implanted at the distal end of a main prosthesis in the common iliac to facilitate placement of at least one leg extension stent graft into the external iliac and hypogastric arteries to maximize blood flow into and minimize obstruction of the hypogastric artery.

The coupler of the invention can be employed as part of a kit of a modular stent graft prosthesis. Insertion of a coupler into the distal end of a main stent graft component of the kit enables the main stent graft component to have a lower profile during delivery of that component to an aneurysm site, relative to the profile of a main stent graft component having luminal grafts seated within the distal end of the stent graft prior to delivery to the aneurysm site.

In addition, the graft prostheses of the invention can be inserted into a previously-implanted prosthesis, in the event that aortic damage has spread, such as when the aneurysm sac has become enlarged, and in the case of AAA repair, to facilitate spanning the aneurysm sac while maximizing blood flow from the hypogastric arteries to surrounding viscera and associated structures. Alternatively the coupler of the invention can be implanted directly into an artery and secured there by suitable means, such as by outwardly protruding barbs.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The same number in different drawings represents the same item. The drawings are not necessarily to scale, emphasis instead being placed upon illustrating embodiments of the present invention.

FIGS. 1A-1C are three-dimensional (3D) (FIG. 1A), and longitudinal-sectional views taken along lines 1B-1B (FIG. 1B) and 1C-1C (FIG. 1C) shown in FIG. 1A, respectively, of one embodiment of a graft prosthesis coupler of the invention, including a circumferential graft, a plurality of luminal grafts, and a first end wall at a concave surface defined by respective first ends of the circumferential graft and luminal grafts.

FIG. 2 is a 3D view of another embodiment of the invention, wherein a plurality of luminal grafts each define a first end, a second end, a lumen and a longitudinal axis, the longitudinal axes of the luminal grafts being substantially parallel to each other, wherein the plurality of the luminal grafts are attached to each other, and wherein at least one of the first ends and the second ends are essentially coplanar.

FIG. 3A is a 3D view and FIG. 3B is an end view of another embodiment of a prosthesis of the invention, wherein one of the luminal grafts has a cross-sectional configuration that is not circular, but, rather, elliptical.

FIGS. 4A-4C are 3D (FIG. 4A), longitudinal-sectional taken along line 4B-4B (FIG. 4B), and end view (FIG. 4C) of another embodiment of a graft prosthesis coupler of the invention, wherein the prosthesis includes four luminal grafts extending within a circumferential graft, and a first end wall lies in a concavity defined by the first end of the circumferential graft and the first ends of the plurality of luminal grafts.

FIG. 5 is a 3D view of a stent graft prosthesis system that includes a graft prosthesis coupler and a plurality of branch grafts.

FIG. 6 is a 3D view of a modular stent graft prosthesis system of the invention that includes a stent graft component, a graft prosthesis coupler, and a plurality of branch grafts.

FIG. 7A is a side view of a stent graft implanted in an abdominal aortic aneurysm as one step of a method of the invention.

FIG. 7B is a 3D view of the stent graft shown in FIG. 7A, following implantation of a graft prosthesis coupler of a modular stent graft prosthesis system of the invention by a method of the invention.

FIG. 7C is a 3D view of the stent graft component and graft prosthesis coupler shown in FIG. 7B, following subsequent implantation of branch grafts within luminal grafts of the graft prosthesis coupler and aortic branches at a distal end of a surgical site of a patient by a method of the invention.

FIG. 8A is a side view of another embodiment of a modular stent graft prosthesis system of the invention, wherein the stent graft component is bifurcated, and wherein the branch grafts each include a cross-sectional diameter that changes along the length of each respective branch graft.

FIG. 8B is a 3D view of the bifurcated stent graft component shown in FIG. 8A following insertion of a graft prosthesis coupler component into a distal end of one leg of the bifurcated stent graft component.

FIG. 8C is a side view of the modular stent graft prosthesis system of FIGS. 8A and 8B, following implantation of the branch grafts into luminal grafts of the graft prosthesis coupler component within the distal end of one leg of the bifurcated stent graft component.

FIG. 9A is a side view of a bifurcated stent graft component following implantation at an aortic aneurysm of a patient according to one embodiment of a method of the invention, wherein a longer leg of the bifurcated stent graft is implanted in one branch of the aorta distal to the aneurysm, and a shorter leg of the bifurcated stent graft component terminates at the aneurysm.

FIG. 9B is a side view of the bifurcated stent graft component shown in FIG. 9A, and a perspective view of a graft prosthesis coupler component of the invention following implantation in the distal end of the shorter leg of the bifurcated stent graft component according to a method of the invention.

FIG. 9C is a side view as in FIG. 9B, following implantation of the proximal end of a branch graft within a luminal graft of the graft prosthesis coupler by a method of the invention, wherein a distal end of the branch graft is secured within an artery distal to the aneurysm site.

FIG. 9D is a side view of a modular stent graft prosthesis system of the invention, following implantation of a second branch graft into a luminal graft of the graft prosthesis coupler and implantation of the distal end of the second branch graft within a distinct artery, also distal to the aneurysm, by a method of the invention.

FIGS. 10A-10C are representations of another embodiment of a modular stent graft prosthesis system of the invention, wherein the modular stent graft prosthesis system includes four branch grafts, rather than two, as shown in FIGS. 9A-9D.

FIG. 11A is a bifurcated stent graft implanted in the aneurysm site of a patient by a method of the invention, wherein a distal end of both legs of the bifurcated stent graft component terminate at the aneurysm site.

FIG. 11B is a representation of the stent graft shown in FIG. 11A, but shown partially in perspective view, wherein a graft prosthesis coupler component of a modular stent graft prosthesis system of the invention has been implanted in the distal end of a shorter leg of the bifurcated stent graft component by a method of the invention.

FIG. 11C is a representation of the stent graft component system of FIGS. 11A and 11B, following implantation of the proximal end of each of four branch grafts within luminal grafts of the graft coupler, and wherein distal ends of the branch grafts have been implanted in respective branch arteries at the aneurysm site, and secured within those branch arteries at respective distal ends of the branch grafts by a method of the invention.

FIG. 11D is a side view of the stent modular stent graft prosthesis system following implantation of a proximal end of an extender graft into the longer leg of bifurcated stent graft and a distal end of the extender graft into the aorta beyond the diseased tissue of the aneurysm, thereby causing the modular bifurcated stent graft prosthesis system to span the aneurysm site by a method of the invention.

### DETAILED DESCRIPTION

The invention is generally directed to prostheses and to methods for their use in treating vascular disease, such as may be beneficial or required during AAA repair when the distal end of the aneurysm is inferior to the hypogastric artery or to encase an enlarged aneurysm sac where a prosthesis has previously been implanted.

The features and other details of the invention, either as steps of the invention or as combinations of parts of the invention will now be more particularly described and pointed out in the claims. It will be understood that the particular embodiments of the invention are shown by way of illustration and not as limitations of the invention. The principle features of this invention can be employed in various embodiments without departing from the scope of the invention.

A description of example embodiments of the invention follows.

When reference is made herein to a prosthesis to be delivered, or implanted in a patient, the word “proximal” means that portion of the prosthesis or component of the prosthesis that is relatively close to the heart of the patient and “distal” means that portion of the prosthesis or component of the prosthesis that is relatively far from the heart of the patient. A “longitudinal axis,” as that term is defined herein, means an axis along a lengthwise direction of a body that also passes through a center of gravity of the body.

When, however, reference is made to a delivery system or a component of a delivery system employed to deliver, or implant, a prosthesis, the word, “proximal,” as employed herein, means closer to the clinician using the delivery system. When reference is made to a delivery system or a component of a delivery system, “distal,” as that term is employed herein, means, further away from the clinician using the delivery system.

For clarity, the word “proximate” means “close to,” as opposed to the meanings ascribed to “proximal” or “distal” described above with respect to either the prosthesis or a delivery system.

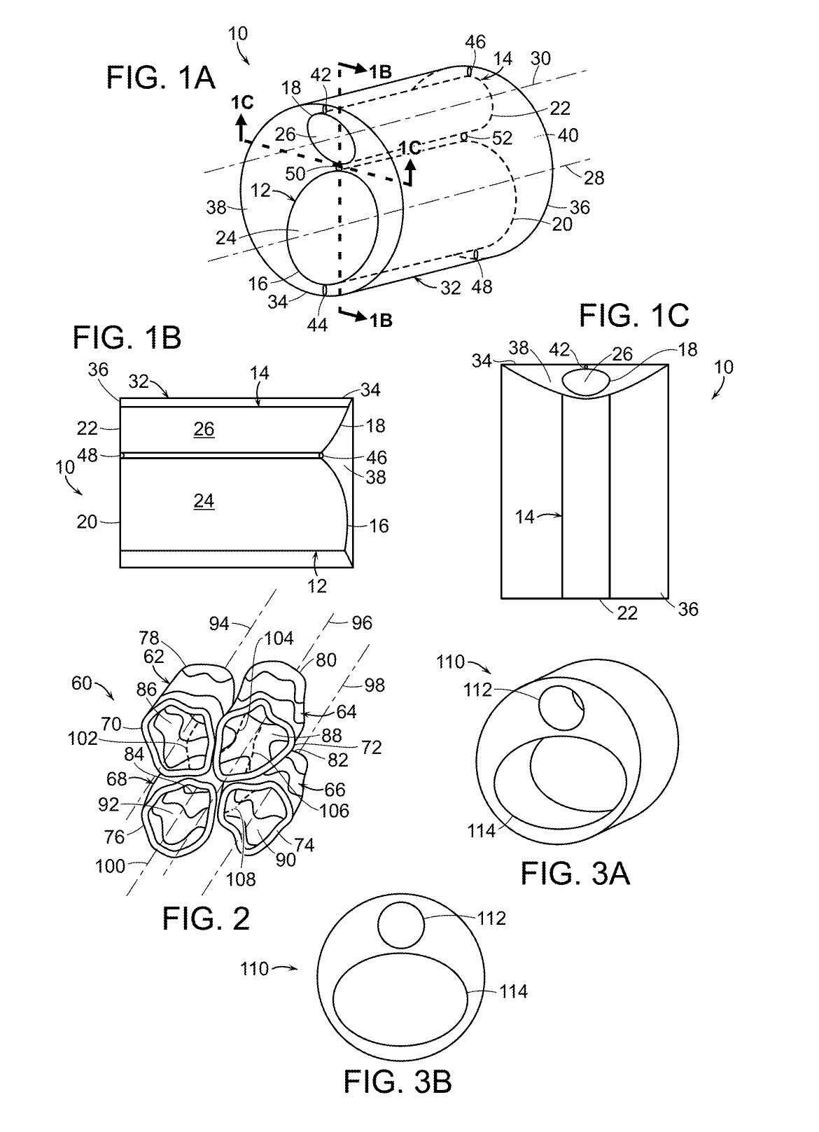

One embodiment of a graft prosthesis coupler 10 of the invention is shown in FIGS. 1A-1C. As shown therein, graft prosthesis coupler 10 includes a plurality of luminal grafts 12,14, each of which defines first end 16,18, second end 20,22, lumen 24,26 between first end 16,18 and second end 20,22, and longitudinal axis 28,30. Longitudinal axes 28,30 of luminal grafts 12,14 are substantially parallel to each other. Circumferential graft 32 extends about the plurality of luminal grafts 12,14 and includes first end 34 and second end 36. First end wall 38 lies in a surface defined by first ends 16,18 of the plurality of luminal grafts 12,14 and first end 34 of circumferential graft 32. Second end wall 40 lies in a surface defined by second ends 20,22 of luminal grafts 12,14 and circumferential graft 32. Luminal grafts 12,14 are fixed to or are part of, first wall 38 and second end wall 40. First and second end walls 38,40, in turn, are fixed to, or are part of, first and second ends 34,36 of circumferential graft 32. Luminal grafts 12,14 can be fixed to first wall 38 and second wall 40, and first and second end walls 38,40 can be fixed to first and secured ends 34,36 of circumferential graft 32 by a suitable means, such as by biocompatible sutures. Examples of suitable sutures include sutures fabricated of polyester ePTFE (expanded polytetrafluoroethylene), polyglycolic acid, polylactic acid, monocryl and polydioxanone, non-absorbable nylon, polyester, PVDF (polyvinylidene difluoride) and polypropylene, or biocompatible adhesive.

In another embodiment, first end 16,18 of at least one of the plurality of luminal grafts 12,14 is secured to circumferential graft 32 by proximal sutures 42,44. Optionally, second end of at least one of the plurality of luminal grafts is secured to circumferential graft by distal sutures 46,48. Alternatively, at least one of the plurality of luminal grafts can be secured to circumferential graft by more than a single suture, such as along an intermittent or continuous length (not shown), over a portion or the entire length of at least one of the plurality of luminal grafts. Luminal grafts 12,14, can also be fixed to each other by sutures 50,52.

Luminal grafts 12,14 and circumferential graft 32 are made from suitable materials, such as are known to those skilled in the art, including, for example, expanded polytetrafluoroethylene (PTFE), such as expanded PTFE (ePTFE), and polyethylene terephthalate (PET), such as woven polyester. Suitable radiographic markers, not shown, such as are known to those skilled in the art, are sutured to at least one of first end or second end of the plurality of luminal grafts, or first end or second end of the circumferential graft.

FIG. 2 is a perspective view of an embodiment of another embodiment of a graft prosthesis coupler of the invention. As shown therein, graft prosthesis coupler 60 includes four luminal grafts 62,64,66,68. Luminal grafts 62,64,66,68 each define first end 70,72,74,76, second end 78,80,82,84, lumen, 86,88,90,92, and longitudinal axis 94,96,98,100. Longitudinal axes 94,96,98,100 are substantially parallel to each other. First ends 70,72,74,76 are coplanar. Second ends 78,80,82,84 are coplanar. Luminal grafts are attached to each other by suitable means, such as sutures 102,104,106,108, as are known in the art.

FIGS. 3A and 3B are a perspective view and an end view, respectively, of yet another embodiment of a graft prosthesis coupler of the invention. As shown therein, prosthesis coupler 110 has two luminal grafts 112,114 that have diameters that differ from each other, and one of which has a cross-section of a substantially non-circular cross-sectional shape. Specifically, luminal graft 112 is circular in cross-section, and luminal graft 114 is elliptical in cross-section.

In a further embodiment, not shown, prosthesis includes three luminal grafts having diameters that are about equal to each other and have a cross-section of a substantially circular cross-sectional shape. In another embodiment, not shown, the three luminal grafts have diameters, at least one of which differs from the others and, optionally each of which independently may have a cross-section that is non-circular, such as an elliptical cross-sectional shape. The three luminal grafts are distributed radially about major longitudinal axis of circumferential graft (not shown), and are, in an embodiment, fixed to each other at respective ends or along a portion or the entire length of the luminal grafts by suitable means, such as sutures or by adhesive.

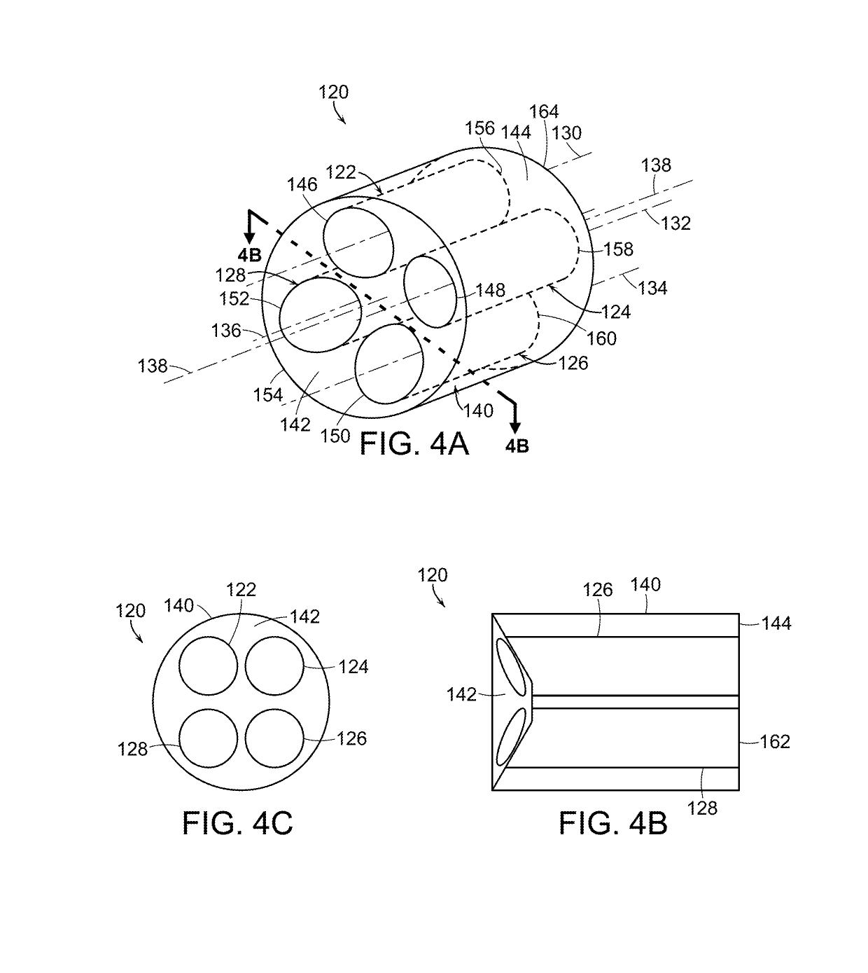

In still another embodiment, shown in FIGS. 4A through 4C, graft prosthesis coupler 120 includes four luminal grafts 122,124,126,128 having diameters that are about equal to each other and have a cross-section of a substantially circular cross-section shape. As can be seen in FIG. 4C, which is a plan view of the embodiment shown in FIGS. 4A and 4B, luminal grafts 122,124,126,128 can be circular in cross-section. In other embodiments, not shown, the four luminal grafts can have diameters and shapes that are the same or differ from at least one of the others, and wherein each, independently, may have a cross-section of a substantially non-circular cross-sectional shape, such as an elliptical shape. As shown in FIGS. 4A and 4C, longitudinal axes 130,132,134,136 of four luminal grafts 122,124,126,128 are distributed radially about central longitudinal axis 138 of circumferential graft 140. Concave first end wall 142 and planar second end wall 144 are part of, or secured or fixed to luminal grafts 122,124,126,128, which can also be part of, or secured or fixed, to each other, by additional means, as described above.

As shown in FIGS. 4A and 4B, first ends 146,148,150,152 of the plurality of luminal grafts and first end 154 of circumferential graft collectively define a concavity. Concave first end wall 142 lies in the concavity. Second ends 156,158,160,162 of the plurality of luminal grafts and second end 164 of circumferential graft 140 together define a plane. Second end wall 144 lies in the plane defined by second end 164 of circumferential graft 140 and second ends 156,158,160,162 of the plurality of luminal grafts 122,124,126,128. It is to be understood that graft prosthesis coupler 120 can, alternatively, be configured so that first end wall 142 has another shape, such as a planar shape, and that second end wall 144, independently, can have another shape, such as that of a concavity.

At least one of the plurality of luminal grafts and circumferential graft can include at least one stent. In an embodiment, circumferential graft includes at least one barb extending radially from at least one stent, in particular a stent located on the outside of circumferential graft (not shown). In another embodiment, graft prosthesis coupler of the invention includes at least one barb extending radially and inwardly from at least one stent of the plurality of luminal grafts (not shown). Stents and barbs are formed of a suitable material, such as is known to those skilled in the art, including stainless steel and shape-memory alloys, such as nitinol. Stents are affixed to luminal grafts and circumferential grafts by suitable means known to those skilled in the art, such as by sewing with biocompatible sutures.

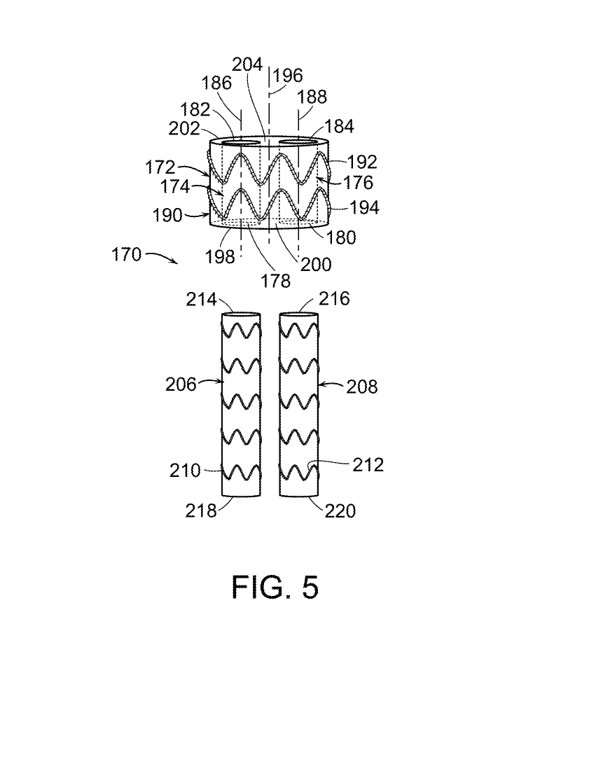

In another embodiment, the invention includes a graft prosthesis coupler and a plurality of branch grafts, such as in a kit. For example, as shown in FIG. 5, modular stent graft prosthesis system 170 includes graft prosthesis coupler 172 having two luminal grafts 174,176, each of which defines first end 178,180 and second end 182,184. Each luminal graft defines longitudinal axis 186,188. Longitudinal axes 186,188 are substantially parallel to each other. Two luminal grafts 174,176 can have cross-sectional diameters that are the same or different from each other. Circumferential graft 190 surrounds luminal grafts 174,176. Stents 192,194 can support circumferential graft 190. In another embodiment, not shown, the modular stent graft prosthesis system can include more than two luminal grafts, which can have cross-sectional diameters that are the same or different from at least one other luminal graft, in any combination. Regardless of the number of luminal grafts, each of the luminal grafts can independently have a cross-section that is circular or is non-circular, such as a cross-section that is elliptical. In one embodiment, longitudinal axes 186,188 of luminal grafts 174,176 are distributed radially about longitudinal axis 196 of circumferential graft 190.

First ends 178,180 of luminal grafts 174,176 and first end 198 of circumferential graft 190 define a concavity, as described above, and in which lies first end wall 200. Second ends 182,184 of luminal grafts 174,176 and second end 202 of coupler 172 together define a plane. In a specific embodiment, modular stent graft prosthesis includes second end wall 204 that lies in the plane between second end 202 of circumferential graft 190 and second ends 182,184 of luminal grafts 174,176. In an embodiment, luminal grafts 174,176 are fixed at first ends 178,180 and second ends 182,184 to first end wall 200 and second end wall 204, respectively, and are part of, or secured or fixed, by suitable means, such as biocompatible sutures or adhesive. First end wall 200 and second end wall 204 are secured to circumferential graft 190 at first end 198 and second end 202, respectively, by suitable means, such as biocompatible sutures or adhesive.

Modular stent graft prosthesis system 170 also includes plurality of branch grafts 206, 208. Each branch graft 206,208 is configured to fit within one of the plurality of luminal grafts 174,176 of graft prosthesis coupler 172. Branch grafts 206,208 each independently include one or more stents 210,212, such as stents formed of nitinol, to provide support for the graft material of the respective graft prosthesis coupler or branch grafts. Use of stents is understood to be optional. Stents 210,212 can include inwardly or outwardly extending barbs, not shown, for securing branch grafts 206,208, either within at least one of luminal grafts 174,176, and within arterial tissue.

Branch grafts 206,208 can have a cross-sectional diameter that varies along the respective lengths. Branch grafts 206,208 include proximal ends 214,216 and distal ends 218,220. In embodiments, not shown, at least one branch graft includes a proximal end having a larger diameter or a smaller diameter than a distal end. It is to be understood that the cross-sectional diameter along each branch graft can vary independently of the cross-sectional diameters along the length of the other branch grafts.

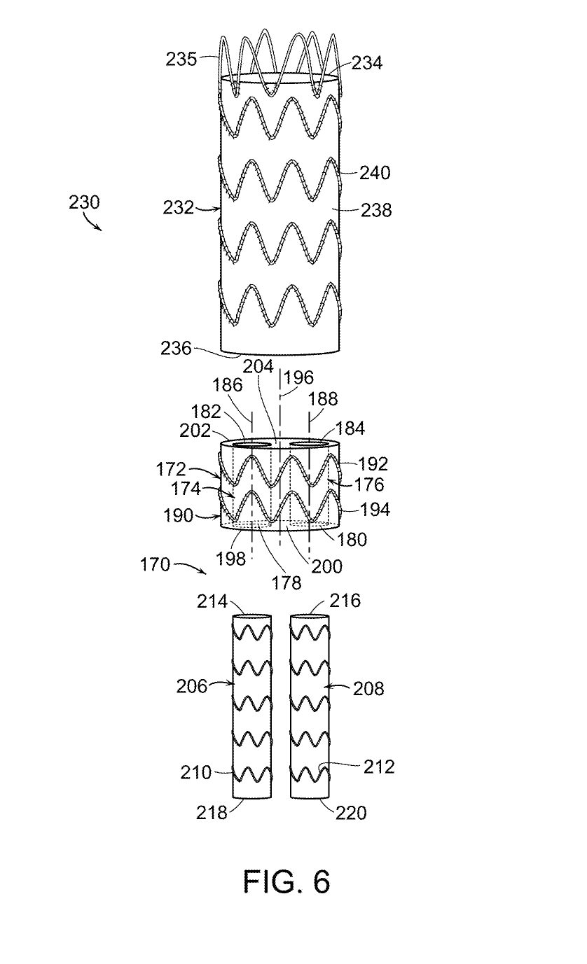

In yet another embodiment, shown in FIG. 6, modular graft prosthesis system 230 includes stent graft 232 in addition to graft prosthesis coupler 172 and branch grafts 206,208. Stent graft 232 includes proximal end 234 and distal end 236. A lumen is defined by graft material 238 extending from first end 234 to second end 236. Graft prosthesis coupler 172 is configured to fit within distal end 236 of stent graft 232. Stents 240 support graft material 238. Bare stent 235 extends proximally from proximal end 234 of stent graft 232.

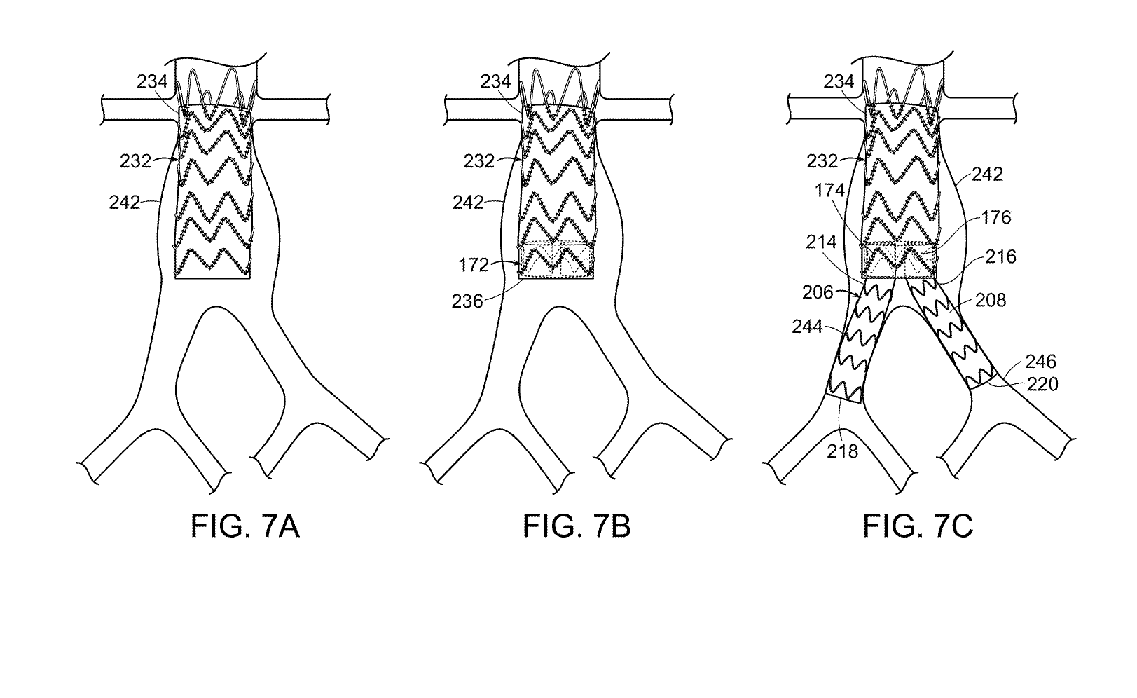

In another embodiment, the invention is a method of implanting a modular stent graft prosthesis of the invention. As shown in FIG. 7A, stent graft 232 is first delivered to aneurysm site 242 of a patient and secured at proximal (or first) end 234 to relatively healthy tissue proximal to aneurysm site 242. Alternatively, stent graft 232 can be an implant previously delivered to aneurysm site 242 of a patient, wherein the aneurysm subsequently extended distally beyond distal (or second) end 236 of stent graft 232. In this instance, the method of the invention includes enhancing a stent graft repair of an aneurysm by extending a pre-existing stent graft 232 with modular stent graft system 170, including graft prosthesis coupler 172 and branch grafts 206, 208, such as from a kit of the invention.

In both embodiments, whether stent graft 232 had previously been implanted at an earlier aneurysm that has since enlarged in size, or is implanted as part of a modular repair of a new aneurysm, graft prosthesis coupler 172 is delivered within distal end 236 of stent graft 232, as shown in FIG. 7B. Thereafter, proximal (or first) end 214,216 of branch grafts 206,208 is inserted into a respective luminal graft 174,176 of graft prosthesis coupler 172, as shown in FIG. 7C. The order of delivery of branch grafts 206,208 is a matter of choice by the surgeon. If not already in place during delivery of the proximal (or first) ends 214,216 of branch grafts 206,208 to luminal grafts 174,176, distal ends 218,220 of branch grafts 206,208 can then be delivered to respective aortic branches 244,246, such as the common iliac arteries, by appropriate means, such as are known to those of skill in the art, thereby completing implantation of modular stent graft prosthesis 170, or 230, to aneurysm site 242 of the patient by a method of the invention.

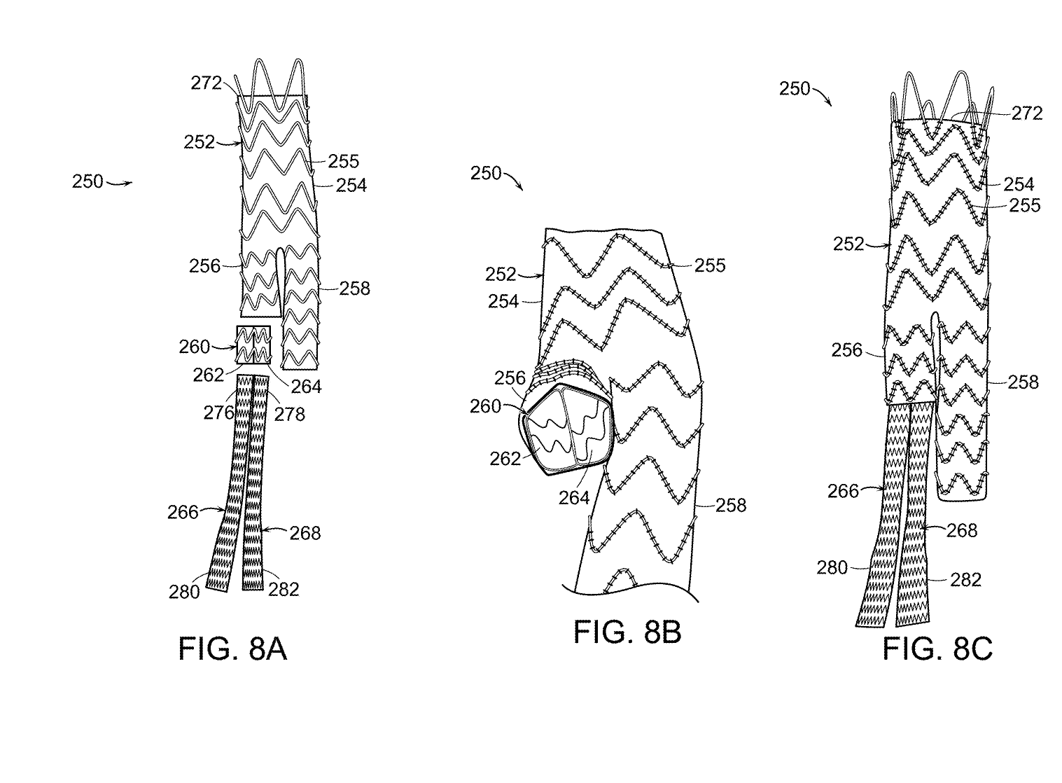

In another embodiment of the invention, modular stent graft prosthesis system 250, shown in FIGS. 8A-8C, includes bifurcated stent graft 252. Bifurcated stent graft 252 includes main stent graft component 254, first leg 256 extending from main stent graft component 254, and second leg 258 extending from main stent graft component 254 and substantially parallel to first leg. Bifurcated stent graft 252 can be supported by stents 255. Graft prosthesis coupler 260 of modular stent graft prosthesis system 250 includes plurality of luminal grafts 262,264, each of which defines a longitudinal axis, wherein the major longitudinal axes are substantially parallel to each other. It is to be understood that, in another embodiment, not shown, graft prosthesis coupler can include a circumferential graft and at least one end wall, as described above. Graft prosthesis coupler 260 is configured to be fit within first leg 256 of bifurcated stent graft 252, as shown in FIG. 8B. Optionally, any space between luminal grafts 262,269 and first leg 256 can be sealed by suitable means known in the art, typically at the proximal ends of luminal grafts 262,264. Optionally, barbs, not shown, can extend radially outward from at least one stent of graft prosthesis coupler. Modular stent graft prosthesis system 250 also includes a plurality of branch grafts 266,268, where each branch graft 266,268 can fit within one of the plurality of luminal grafts 262,264 of graft prosthesis coupler 260, as shown in FIG. 8C.

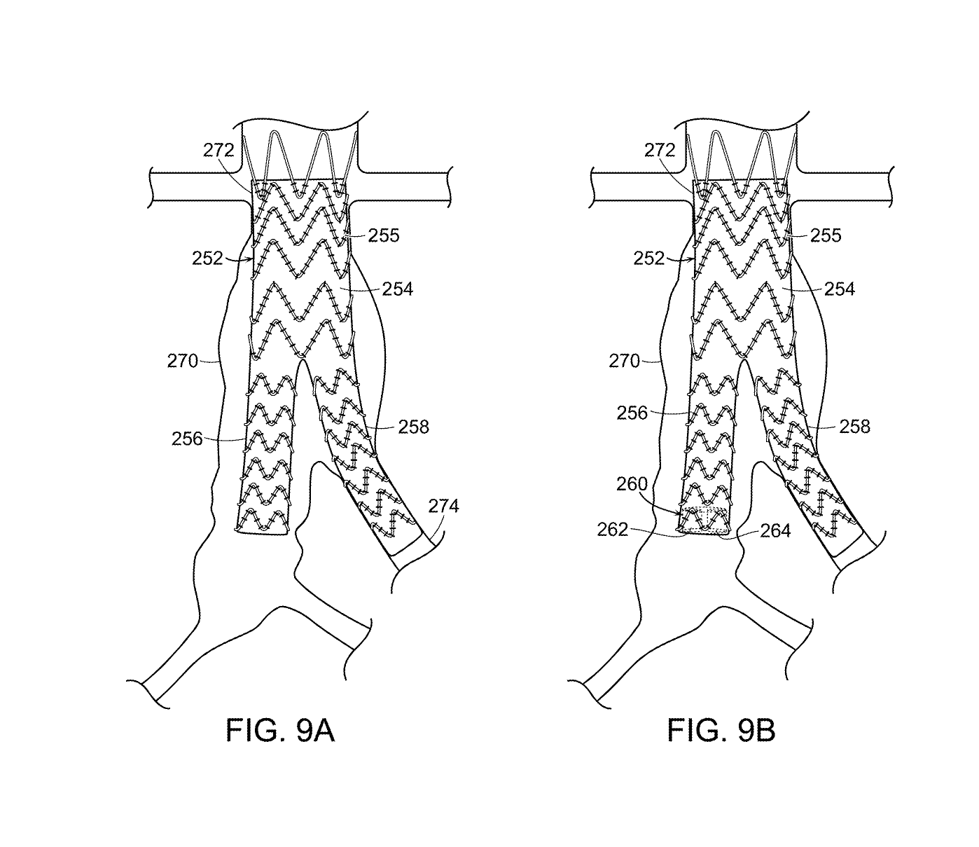

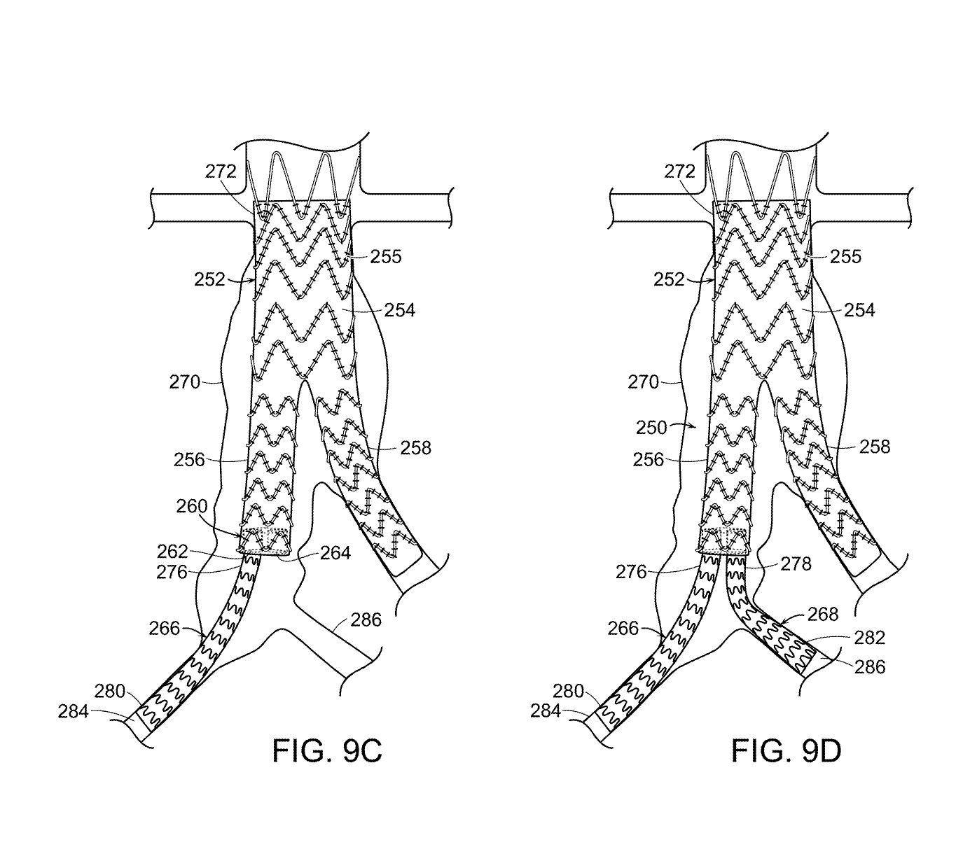

In one embodiment of a method of the invention, modular stent graft prosthesis system 250, is implanted at the thoraco-abdominal aneurysm site 270, as shown in FIG. 9A. In one specific embodiment, bifurcated stent graft 252 is delivered to aneurysm site 270, whereby proximal end 272 of bifurcated stent graft 252 is secured to relatively healthy tissue of a blood vessel proximal to aneurysm site 270. In this embodiment, second leg 258 of bifurcated stent graft 252 is secured within aortic branch 274, such as a common iliac artery, at aneurysm site 270. Alternatively, bifurcated stent graft 252 is previously existing from an earlier surgical repair of an aneurysm that has since enlarged distally beyond shorter leg (or first branch) 256 of bifurcated stent graft 252. Graft prosthesis coupler 260 is delivered within first branch 256 of bifurcated stent graft 252, as shown in FIG. 9B, followed by delivery of branch grafts 266,268 to respective luminal grafts 262,264 of graft prosthesis coupler 260, and secured therein, as shown in FIGS. 9C and 9D. Proximal end 272 of bifurcated stent graft 252, second branch 258 of bifurcated stent graft 252, graft prosthesis coupler 260 and proximal ends of luminal grafts 262,264, respectively, can each independently be secured by use of barbs extending radially from respective stents in each component, as appropriate, by suitable means, such as are known in the art. Distal ends 280,282 of branch grafts 266,268, if not already placed within respective aortic branches 284,286 during delivery of the respective proximal ends 276,278 of the branch grafts 266,268, are then delivered and secured to respective aortic branches 284,286 such as the external iliac artery and internal iliac artery (also knowns as the hypogastric artery), to complete implantation of the modular stent graft prosthesis 250 of the invention.

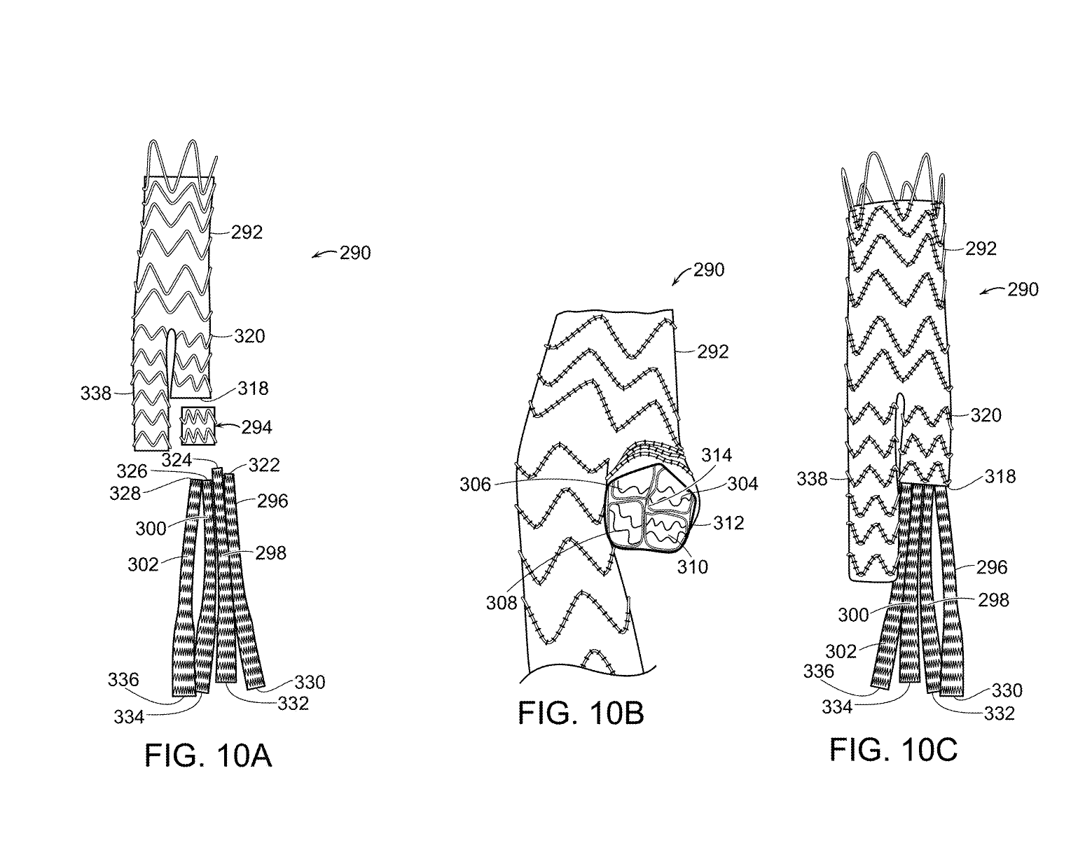

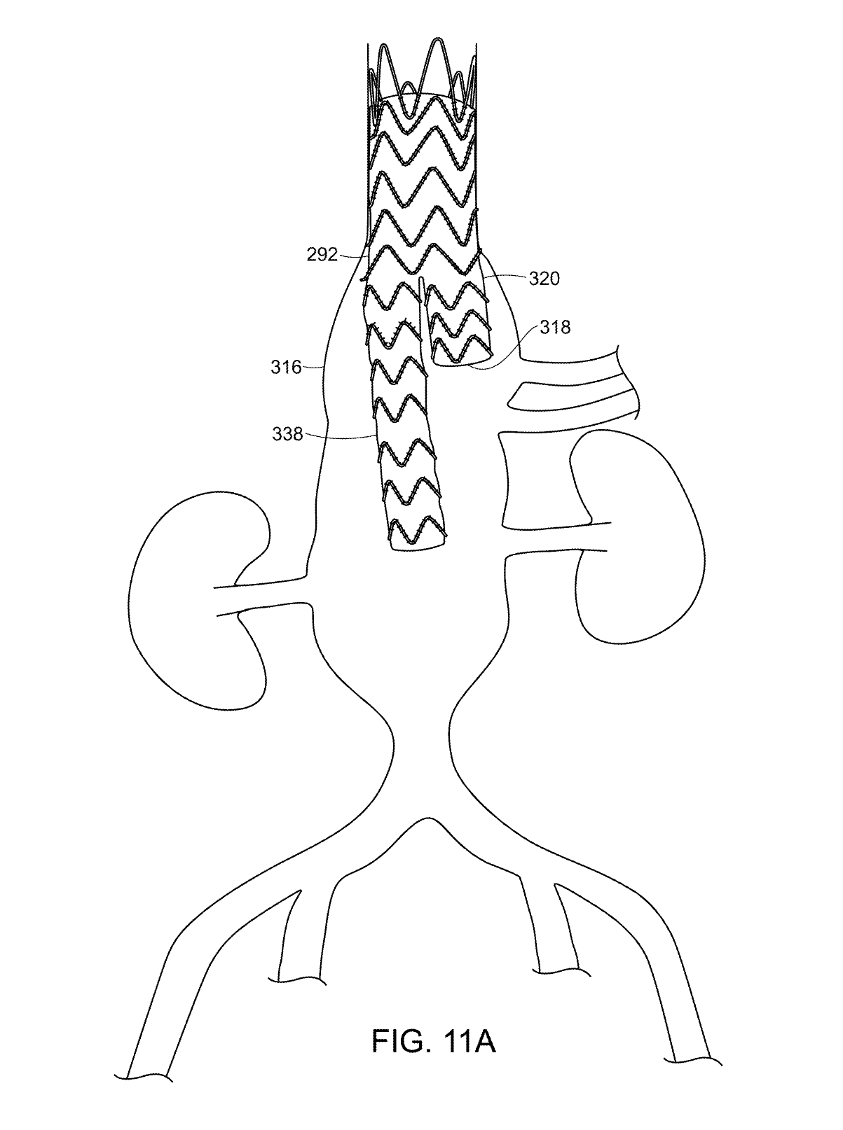

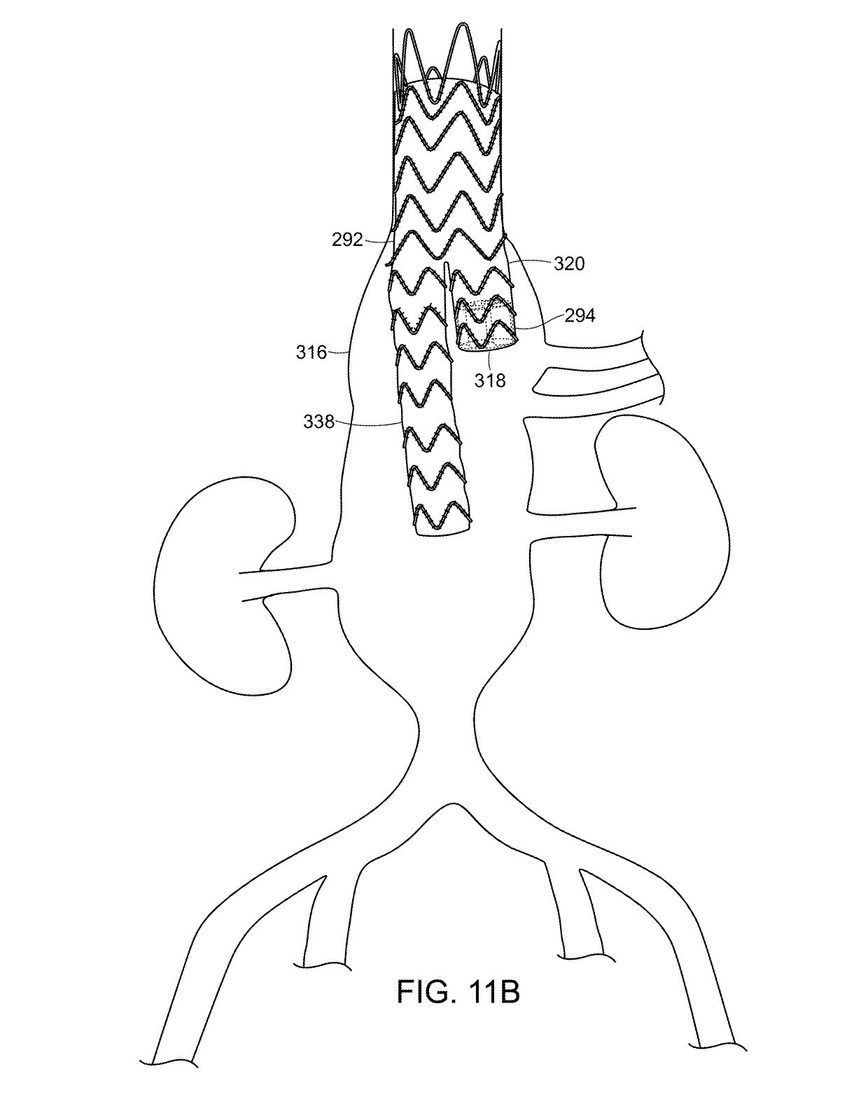

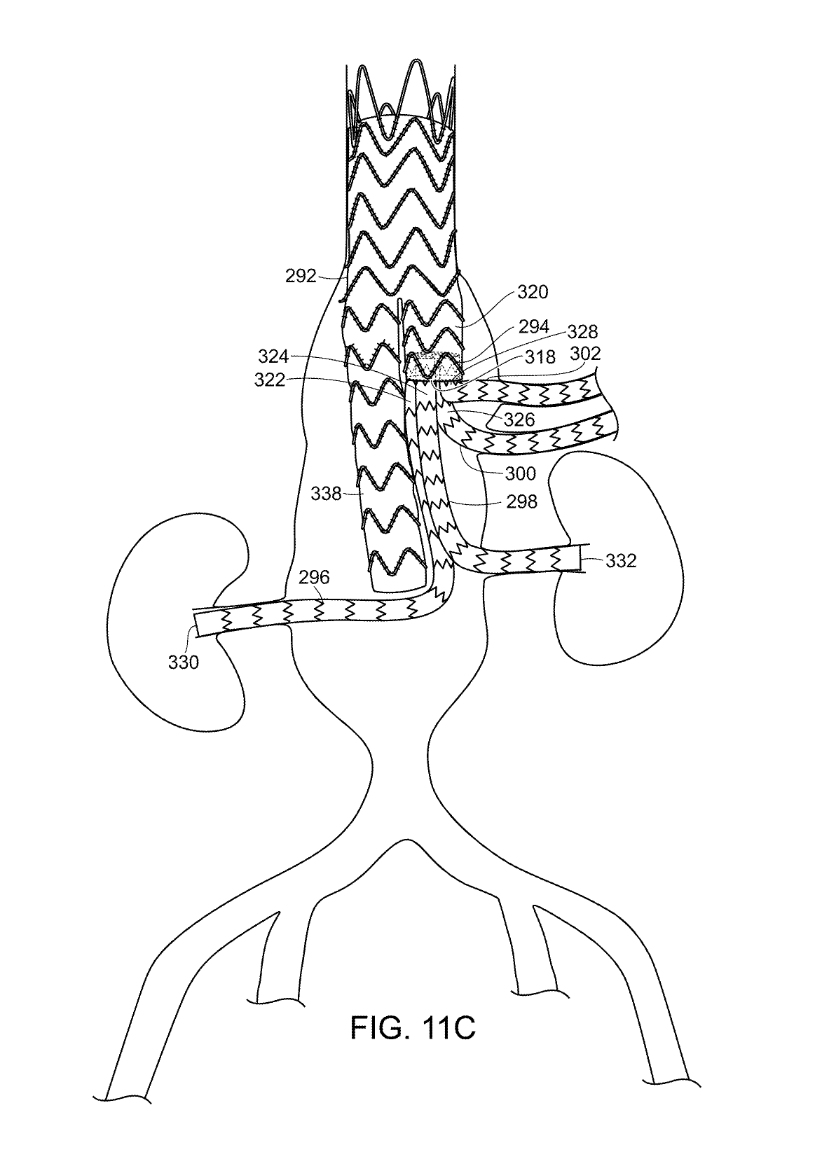

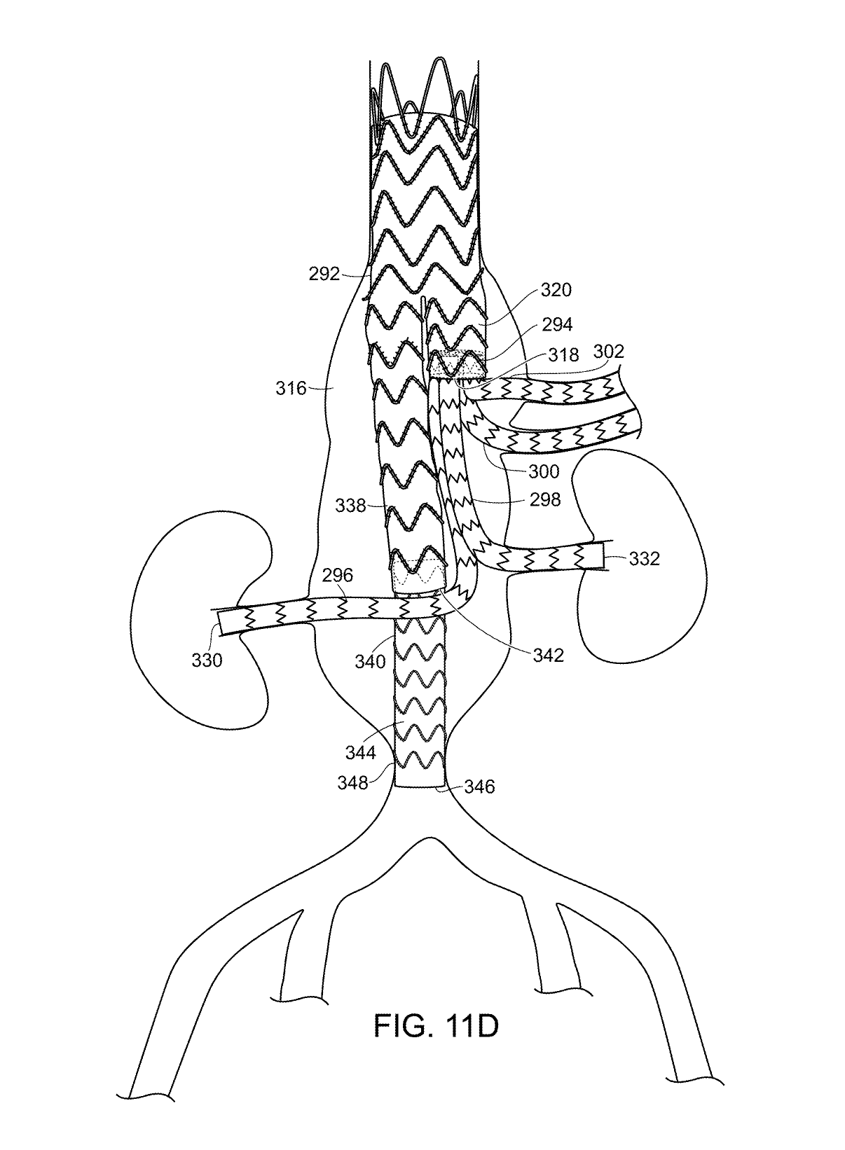

In yet another embodiment of the invention, shown in FIGS. 10A through 10C, modular stent graft prosthesis system 290 includes bifurcated stent graft 292, graft prosthesis coupler 294, and four branch grafts 296,298,300,302, all having a construction that is the same as, or similar to, the component parts of embodiments of the invention discussed above. For example, as shown in FIGS. 10A and 10B, graft prosthesis coupler 294 includes four luminal grafts 304,306,308,310, circumferential graft 312 and first end wall 314. In one method for implanting the embodiment shown in FIGS. 10A-C, bifurcated stent graft 292 is first implanted in aortic aneurysm 316 of a patient, as shown in FIG. 11A. It is to be understood that bifurcated stent graft 292 may have previously been implanted, wherein the method of the invention is extension, or enhancement, of previously implanted (referred to as “pre-existing”) bifurcated stent graft 292 by delivery of graft prosthesis coupler 294 and branch grafts 296,298,300,302. Graft prosthesis coupler 294 is then delivered to distal end 318 of first leg 320, as shown in FIG. 11B. Proximal ends 322,324,326,328 of branch grafts are then each delivered to a luminal graft of coupler, and distal ends of branch graft are then delivered to respective aortic branches, as shown in FIG. 11C.

Leg extension graft 344, shown in FIG. 11D, may be a component of a kit of the invention, either with or without stent graft 292. Proximal end 340 of leg extension graft 344 is delivered to distal end 342 of second leg 338 of bifurcated stent graft 292. Distal end 346 of leg extension stent graft 344 is secured within healthy tissue beyond distal end 348 of aneurysm 316, as shown in FIG. 11D. The order of delivery of extension and branch grafts is a matter of choice, depending upon the surgeon and the nature of the aneurysm.

In another embodiment, the graft prosthesis coupler of the invention can be implanted directly into an artery and secured there by suitable means, such as by outwardly protruding barbs (not shown). Once the graft prosthesis coupler is implanted into an artery, such as at a site proximal or distal to an aneurysm site, branch graft prostheses can be inserted at one end into a corresponding one of the plurality of luminal grafts of the graft prosthesis coupler and at the other end into a corresponding arterial branch of a patient to implant a modular graft prosthesis at an aneurysm site of a patient (not shown).

Means for securing each of the components within the patient can be, for example, by barbs, or such other means as are appropriate, as are known in the art. Securement of distal ends of branch grafts and extension completes the method of extending a stent graft prosthesis at an aneurysm site of a patient by one method of the invention.

Prostheses of the invention can be implanted, for example, by transfemoral access. Additional vascular repair devices that are directed into the prostheses of the invention can be implanted, for example, by supraaortic vessel access (e.g., through the subclavian artery), or by transfemoral access or other arterial access.

The teachings of all patents, published applications and references cited herein are incorporated by reference in their entirety. The relevant teachings of all patents, published applications and references cited herein are incorporated by reference in their entirety. The relevant teachings of U.S. Pat. Nos. 8,292,943; 7,763,063; 8,308,790; 8,070,790; 8,740,963; 8,007,605; 9,320,631; 8,062,349; 9,198,786; 8,062,345; 9,561,124; 9,173,755; 8,449,595; 8,636,788; 9,333,104; 9,408,734; 9,408,735; 8,500,792; 9,220,617; 9,364,314; 9,101,506; 8,998,970; 9,554,929; 9,439,751; 9,592,112 and 9,655,712; U.S. patent application Ser. Nos. 14/226,005; 14/575,673; 15/166,818; 15/167,055; 14/272,818; 14/861,479; 15/478,424; 15/478,737; 15/604,032 and PCT/US2017/025849 are also incorporated by reference in their entirety.

While example embodiments have been particularly shown and described, it will be understood by those skilled in the art that various changes in form and details may be made therein without departing from the scope of the embodiments encompassed by the appended claims.

1. A graft prosthesis coupler comprising:
a) a plurality of luminal grafts, each of which defines a first end, a second end, an outside surface between the first end and the second end, and a longitudinal axis, the longitudinal axes being substantially parallel to each other;
b) a circumferential graft that extends about and secures the plurality of luminal grafts relative to each other, the circumferential graft having a first end and a second end; and
c) a first end wall that lies in a surface defined by the first ends of the luminal grafts and the first end of the circumferential graft.

2. The graft prosthesis coupler of claim 1, including two luminal grafts.

3. The graft prosthesis coupler of claim 2, wherein the two luminal grafts have diameters that are different from each other.

4. The graft prosthesis coupler of claim 1, including four luminal grafts.

5. The graft prosthesis coupler of claim 4, wherein the four luminal grafts have diameters that are about equal to each other.

6. The graft prosthesis coupler of claim 5, wherein the longitudinal axis of the four luminal grafts are distributed radially about a longitudinal axis of the circumferential graft.

7. The graft prosthesis coupler of claim 1, wherein at least one of the luminal grafts has a cross-section that has a non-circular cross-sectional shape.

8. The graft prosthesis coupler of claim 1, wherein the first ends of the luminal grafts and the circumferential graft collectively define a concavity.

9. The graft prosthesis coupler of claim 1, wherein the circumferential graft includes at least one stent.

10. The graft prosthesis coupler of claim 1, wherein at least one of the luminal grafts includes at least one stent.

11. The graft prosthesis coupler of claim 9, wherein the circumferential graft includes at least one barb extending radially from at least one of the at least one stent.

12. The graft prosthesis coupler of claim 1, wherein the second ends of the plurality of luminal grafts and the circumferential graft together define a plane.

13. The graft prosthesis coupler of claim 12, further including a second end wall that lies in the plane between the circumferential graft and the luminal grafts.

14. The graft prosthesis coupler of claim 1, further including at least one barb extending radially and inwardly from at least one of the plurality of luminal grafts.

15. A modular graft prosthesis system, comprising:
a) a graft component having a proximal end and a distal end;
b) a graft prosthesis coupler, including
i) a plurality of luminal grafts, each of which defines a first end, a second end, and a longitudinal axis, the longitudinal axes being substantially parallel to each other;
ii) a circumferential graft that extends about and secures the plurality of luminal grafts relative to each other, the circumferential graft having a first end and a second end; and
iii) a first end wall that lies in a surface defined by the first ends of the luminal grafts and the first end of the circumferential graft, and
c) a plurality of branch grafts, wherein each branch graft is conformable to one of the plurality of luminal grafts of the graft prosthesis coupler.

16.-33. (canceled)

34. A modular graft prosthesis system, comprising:
a) a graft prosthesis coupler, including
i) plurality of luminal grafts, each of which defines a first end, a second end, and a longitudinal axis, the longitudinal axes being substantially parallel to each other,
ii) a circumferential graft that extends about the plurality of luminal grafts relative to each other, the circumferential graft having a first and a second end, and
iii) a first end wall that lies in a surface defined by the first ends of the plurality of luminal grafts and the first end of the circumferential graft; and
b) a plurality of branch grafts, wherein each branch graft is conformable to one of the plurality of luminal grafts of the graft prosthesis coupler.

35.-48. (canceled)

49. A graft prosthesis coupler, comprising a plurality of luminal grafts, each of which defines a first end, a second end, a lumen, and a longitudinal axis, the longitudinal axes of the luminal grafts being substantially parallel to each other, wherein the plurality of luminal grafts are attached to each other, and wherein one of the first ends and second ends are substantially coplanar.

50. A method of implanting a modular graft prosthesis at an aneurysm site of a patient, comprising the steps of:
a) delivering a luminal graft through a blood vessel to an aneurysm site in a patient, the luminal graft defining an open proximal end, an open distal end, and a main lumen, the main lumen extending between the open proximal end and the open distal end;
b) inserting a graft prosthesis coupler into the main lumen of the luminal graft, the graft prosthesis coupler including:
i) a plurality of luminal grafts, each of which has a first end and a second end, and defines a longitudinal axis, the longitudinal axes of the luminal grafts being substantially parallel to each other;
ii) a circumferential graft that extends about the plurality of luminal grafts relative to each other, the circumferential graft having a first end and a second end; and
iii) a first end wall that lies in a surface defined by the first ends of the luminal grafts and the first end of the circumferential graft;
c) inserting each of a plurality of branch graft prostheses into a corresponding one of the plurality of luminal grafts of the graft prosthesis, each of the plurality of branch graft prostheses having a branch graft prosthesis open proximal end and a branch graft prosthesis open distal end, wherein the luminal graft, the graft prosthesis coupler and the plurality of branch graft prostheses together make up the modular graft prosthesis; and
d) inserting the distal end of each of the plurality of branch graft prostheses into a corresponding aortic branch of the patient at the aneurysm site, thereby implanting the modular graft prosthesis at the aneurysm site of the patient.

51.-53. (canceled)

54. A method of extending a pre-existing luminal prosthesis at an aneurysm site of a patient, comprising the steps of:
a) delivering a graft prosthesis coupler into a main lumen of a pre-existing luminal prosthesis in a blood vessel at an aneurysm site of a patient, the graft prosthesis coupler including
i) a plurality of luminal grafts, each of which defines a first end and a second end, and a longitudinal axis, the longitudinal axes of the luminal grafts being substantially parallel to each other,
ii) a circumferential graft that extends about the plurality of luminal grafts relative to each other, the circumferential graft having a first end and a second end,
iii) a first end wall that lies in a surface defined by the first ends of the luminal grafts and the first end of the circumferential graft, and
iv) a second end wall that lies in a surface defined by the second ends of the luminal grafts and the second end of the circumferential graft;
b) inserting each of a plurality of branch graft prostheses into a corresponding one of the plurality of luminal grafts of the graft prosthesis coupler, each of the plurality of branch graft prostheses having a branch graft prosthesis open proximal end and a branch graft prosthesis open distal end, the graft prosthesis coupler and the plurality of branch graft prostheses collectively making up the extension of the pre-existing luminal prosthesis; and
c) inserting the distal end of each of the plurality of branch graft prostheses into a corresponding aortic branch of the patient at the aneurysm site, thereby extending the modular stent graft prosthesis at the aneurysm site of the patient.

55.-57. (canceled)

58. A method of implanting a modular graft prosthesis at an aneurysm site of a patient, comprising the steps of:
a) delivering a luminal graft through a blood vessel to an aneurysm site in a patient, the luminal graft defining an open proximal end, an open distal end, and a main lumen, the main lumen extending between the open proximal end and the open distal end;
b) inserting a graft prosthesis coupler through the main lumen of the luminal graft, the graft prosthesis coupler including a plurality of luminal grafts, each of which defining a longitudinal axis, the longitudinal axes of the graft prosthesis couplers being substantially parallel to each other, and wherein the plurality of luminal grafts are attached to each other;
c) inserting each of a plurality of branch graft prostheses into a corresponding one of the plurality of luminal grafts of the graft prosthesis coupler, each of the plurality of branch graft prosthesis having a branch graft prosthesis open proximal end and a branch graft prostheses open distal end, the luminal graft, the graft prosthesis coupler, and the plurality of branch graft prostheses together making up the modular graft prosthesis; and
d) inserting the distal end of each of the plurality of branch graft prostheses into a corresponding arterial branch of the patient at the aneurysm site, thereby implanting the modular graft prosthesis at the aneurysm site of the patient.

59.-61. (canceled)

62. A method of extending or enhancing a pre-existing graft prosthesis at an aneurysm site of a patient, comprising the steps of:
a) delivering a graft prosthesis coupler into a main lumen of a pre-existing luminal graft prosthesis in an aortic blood vessel at an aneurysm site of a patient, the graft prosthesis coupler including,
i) a plurality of luminal grafts, each of which defines a first end and, a second end, and
ii) a longitudinal axis, the longitudinal axes of the luminal grafts being substantially parallel to each other;
b) inserting each of a plurality of branch graft prostheses into a corresponding one of the plurality of luminal grafts of the graft prosthesis coupler, each of the plurality of branch graft prostheses having an open proximal end and an open distal end, the luminal graft, the graft prosthesis coupler and the plurality of branch graft prostheses collectively making up the extension of the pre-existing prostheses; and
c) inserting the distal end of each of the plurality of branch graft prostheses into a corresponding aortic branch of the patient at the aneurysm site, thereby extending the modular stent graft prosthesis at the aneurysm site of the patient.

63.-65. (canceled)
